(19) United States
(12) Patent Application Publication
(10) Pub. No.: US 2010/0094352 A1
(43) Pub. Date: Apr. 15, 2010

(54) BONE SCREW

(76) Inventors: Andrew Iott, Villanova, PA (US); Khiem Pham, Chalfont, PA (US); Jeffrey M. Reuben, Beaufort, SC (US)

Correspondence Address: GLOBUS MEDICAL, INC.;ATTN: BRIAN MALM, VALLEY FORGE BUSINESS CENTER, 2560 GENERAL ARMISTEAD AVENUE, AUDUBON, PA, 19403, US

(21) Appl. No.: 12/249,526

(22) Filed: Oct. 10, 2008

(51) Int. Cl. A61B 17/04

(52) U.S. Cl. 606/301; 606/305; 606/308; 606/316

(57) ABSTRACT

The present invention provides a bone screw which can be used for fixation and/or fastening of prosthetic devices or instruments to bone tissue whose structure or dimensions differ from one region to another. In particular, the present invention provides a bone screw which is designed to optimize purchase in both the cancellous and cortical regions of a vertebral body. In an exemplary embodiment, the bone screw has a distal portion and a proximal portion in which the diameter of the thread on the proximal portion of the screw is greater than the diameter of the thread on the distal portion of the screw.

### FIELD OF THE INVENTION

The present invention relates to the field of orthopedic surgery and more specifically to a bone screw for orthopedic use.

### BACKGROUND OF THE INVENTION

As is known in the field of orthopedic surgery, and more specifically spinal surgery, bone screws may be used for fixation or for the fastening of prosthetic devices or instruments to bone tissue. An exemplary use of bone screws may include using bone screws to fasten a prosthetic device, such as a bone plate or a spinal spacer, to a vertebral body for the treatment of a defect in a patient's spine, such as a fracture within a vertebral body or a degenerating intervertebral disc. Focusing on the bone plate example, bone screws can be used to fasten anchors to a number of vertebral bodies and a bone plate can then be connected to the vertebral bodies using the anchors to fuse a segment of the spine. In another exemplary use, bone screws can be used to fix the location of a spinal spacer once the spacer is implanted between adjacent vertebral bodies.

The bone tissue that comprises the vertebral body, in terms of mechanical characteristics, can be divided into two distinct regions, namely, cancellous bone tissue, which is characterized by voids and a low density, and cortical bone tissue, which is a higher density, stronger bone region. Since the cortical bone tissue region is stronger than the cancellous bone tissue, the cortical bone tissue is better able to support a secure connection for screw fixation than the cancellous bone tissue.

As such, there exists a need for a bone screw that is able to optimally purchase bone tissue where the mechanical characteristics of the bone tissue vary from one region to another to improve fixation and/or fastening of prosthetic devices or instruments to bone tissue.

### SUMMARY OF THE INVENTION

The present invention provides a bone screw which can be used for fixation and/or fastening of prosthetic devices or instruments to bone tissue whose structure or dimensions differ from one region to another. In particular, the present invention provides a bone screw which is designed to optimize purchase in both the cancellous and cortical regions of the vertebral body. In an exemplary embodiment, the bone screw has a distal portion and a proximal portion, each portion having an approximately constant diameter over a portion of its length, in which the diameter of the thread on the proximal portion of the screw is greater than the diameter of the thread on the distal portion of the screw.

Further areas of applicability of the present invention will become apparent from the detailed description provided hereinafter. It should be understood that the detailed description and specific examples, while indicating the preferred or exemplary embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of an exemplary embodiment of the bone screw according to the present invention;

FIG. 2 is a side perspective view of the bone screw shown in FIG. 1;

FIG. 3 is an enlarged partial cross-sectional view of the bone screw shown in FIG. 1; and

FIG. 4 is an enlarged partial cross-sectional second side view of the bone screw shown in FIG. 1.

### DETAILED DESCRIPTION OF THE PREFERRED EMBODIMENTS

The following description of the preferred embodiment(s) is merely exemplary in nature and is in no way intended to limit the invention, its application, or uses.

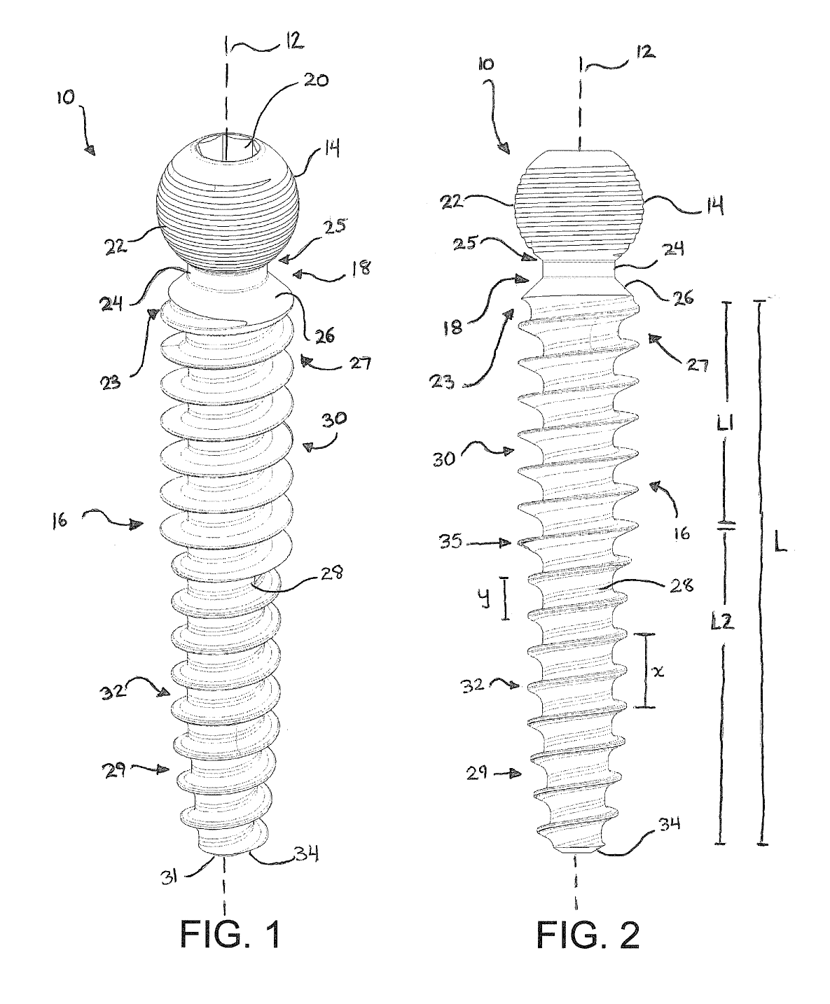

With reference to FIGS. 1 and 2, a preferred embodiment of a bone screw 10 according to the present invention is illustrated. The bone screw 10 preferably includes, concentric to a longitudinal axis 12, a head portion 14, a neck portion 18 and a shank portion 16. The head portion 14 connects to the shank portion 16 through the neck portion 18. The bone screw 10 is preferably constructed from any biocompatible material including, but not limited to, stainless steel alloys, titanium, titanium based alloys, or polymeric materials.

In a preferred embodiment, the head portion 14 of bone screw 10 has a generally spherical shape and includes a recess 20 for receiving a driving instrument. As is well known in the art, the recess 20 may be configured and dimensioned to any shape that corresponds with the end of the driving instrument designed to engage the bone screw 10. For example, the recess 20 may be any one of the following shapes: slot, cross, polygon, or multi-lobes. The generally spherical shape of the head portion 14 is configured and dimensioned to be received within a correspondingly shaped cavity in a receiving member (not shown) which may be part of a spinal fixation system. The shape of the head portion 14 allows the bone screw 10 to pivot, rotate and/or move with respect to the receiving member. In an exemplary use, the head portion 14 of the bone screw 10 is received in the cavity of the receiving member and the bone screw 10 is pivoted, rotated or moved until the desired orientation with respect to the receiving member is met. The bone screw 10 is then locked in place in the cavity of the receiving member. In a further preferred embodiment, the head portion 14 also includes texturing 22 that extends along at least a portion of the head portion 14. The texturing 22 on the head portion 14 provides additional frictional surfaces which aid in locking the bone screw 10 in place with respect to the receiving member.

With continued reference to FIGS. 1 and 2, in a preferred embodiment, the neck portion 18 of the bone screw 10 integrally connects the head portion 14 with the shank portion 16. Preferably, the neck portion 18 includes a generally cylindrical region 24 and a truncated generally frustoconical region 26. The diameter of the distal end 23 of the frustoconical region 26 is preferably dimensioned to match a major diameter (discussed below) of the bone screw 10 while the diameter of the proximal end 25 of the frustoconical region 26 is preferably dimensioned to match the diameter of the generally cylindrical region 24 of the neck portion 18. In a preferred embodiment, the generally cylindrical region 24 will have a diameter that is at least as large as a minor diameter (discussed below) of the bone screw 10, but the diameter of the generally cylindrical region 24 can be smaller than the minor diameter of the bone screw 10. By having the diameter of the neck portion 18 dimensioned at least as large as a minor diameter of the bone screw 10, the overall rigidity and strength of the bone screw 10 is increased.

Turning to FIGS. 1-4, in a preferred embodiment, the shank portion 16 of the bone screw 10 includes a shaft 28, having a length L, surrounded at least in part by a plurality of thread portions 30, 32. The diameter of the shaft 28 is the minor diameter of the bone screw 10 and the diameter of the shaft 28 including the thread portions 30, 32 is the major diameter of the screw 10. In a preferred embodiment, the diameter of the shaft 28 remains generally constant from the proximal end 27 toward the distal end 29 of the shaft 28. However, the diameter of the distal end 29 of the shaft 28 preferably decreases towards the distal tip 34 of the bone screw 10. The constant diameter of a majority portion of the shaft 28 allows for optimal screw positioning when the bone screw is inserted into a predetermined area in the bone tissue. The constant diameter also allows for varying the depth positioning of the bone screw in the bone. For example, if a surgeon places the bone screw 10 into bone tissue at a first depth and decides the placement is more optimal at a second, shallower depth, the bone screw 10 can be backed out to the second depth and still remain fixed in the bone. In another embodiment, the diameter of the shaft 28 may vary along its length, including increasing in diameter from the proximal end to the distal end or decreasing in diameter from the proximal end to the distal end.

Looking at FIGS. 1-2, the plurality of threads 30, 32 surrounding the shaft 28 extend, in a preferred embodiment, from the distal tip 34 of the shaft 28 to the distal end 23 of the frustoconcial region 26 of the neck portion 18. In another preferred embodiment, the threads 30, 32 may extend along only a portion of shaft 28. As seen in FIGS. 1-2, the thread portions 30, 32 are preferably a Modified Buttress thread but the threads can be any other type of threading that is anatomically conforming, including, but not limited to Buttress, Acme, Unified, Whitworth and B&S Worm threads.

In a preferred embodiment, the diameter or depth or height (hereinafter, diameter) of the thread portion 30 remains substantially constant over its length L1 and the diameter of the thread portion 32 remains substantially constant over a portion of its length L2. Preferably, the diameter of the thread portion 32 decreases towards the distal tip 34 of the bone screw 10. By having a decreased diameter thread portion 32 near the distal tip 34 of the bone screw 10, the bone screw 10 can be self-starting. In another preferred embodiment, bone screw 10 may also include at least one flute to clear any chips, dust, or debris generated when the bone screw 10 is implanted into bone tissue.

In a preferred embodiment, the thread portion 30 also differs dimensionally from the thread portion 32. More specifically, the thread portion 30 preferably has a larger diameter than the thread portion 32. The diameter of the thread portion is determined by subtracting the minor diameter from the major diameter of the bone screw 10. For example, if the minor diameter of the bone screw 10 is 4 mm and the major diameter of the bone screw 10 near the proximal end 27 of the shaft 16 is 7 mm, the diameter of the thread portion 30 around the proximal end 27 of the shaft 16 is 3 mm. A preferred difference in the diameter between the thread portion 30 and the thread portion 32 is 2.0 mm but a larger or smaller difference between the thread portion diameters is also contemplated. In a preferred embodiment, the ratio of the diameter of the thread portion 30 to the ratio of the thread portion 32 is approximately 1.2, but can vary from 1.0 to 1.5

By having a larger diameter thread portion 30 and a smaller diameter thread portion 32, the bone screw 10 can grip bone tissue having regions with varying mechanical characteristics in an optimal manner. The larger diameter thread portion 30, which surrounds the proximal portion 27 of the shaft 16, is better suited to grip the cancellous region of the bone. The larger diameter thread portion, having the larger threads and increased purchasing surface area, better engages the softer, less dense bone tissue. Correspondingly, the smaller diameter thread portion 32, which surrounds the distal portion 29 of the shaft 16, is better suited to grip the cortical region of the bone. Since the cortical region is harder and denser, a smaller thread is preferred for the bone screw 10 to optimally purchase that bone tissue region. The combination of the larger diameter thread portion 30 and the smaller diameter thread portion 32 provides for an improved bone screw having greater bone tissue purchasing as well as greater pull-out strength than a screw with a single diameter thread. In a preferred embodiment, the improved purchasing lowers the bone screw 10 toggling over time and the pull out-strength of bone screw 10 compared to a screw having a single outer diameter thread has been determined to be at least 20% higher than the pull-out strength of the screw having a single outer diameter thread.

In a preferred embodiment, the bone screw 10 also has a transition portion 35 between thread portions 30, 32 to allow for easier insertion of the bone screw 10 in the bone tissue. Preferably, over the transition portion 35, the major diameter of the bone screw 10 decreases gradually between the thread portion 30 and thread portion 32 when viewed from a proximal to distal direction.

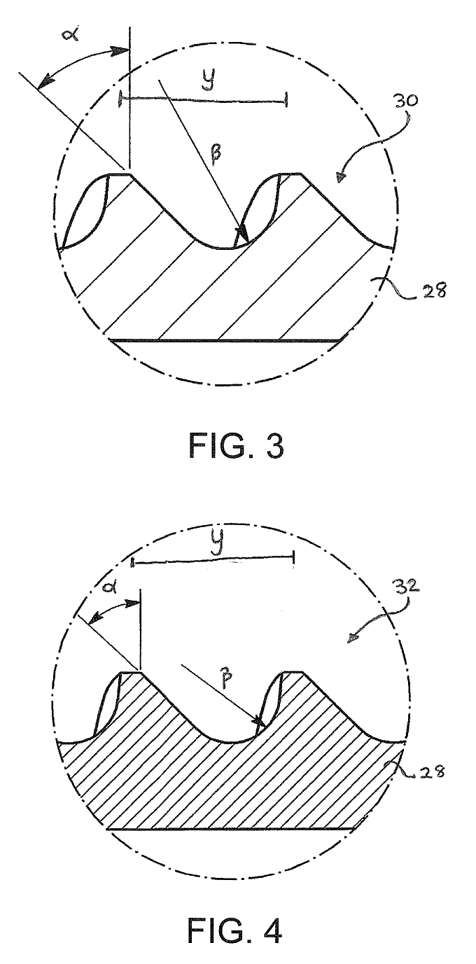

Looking now at FIGS. 3-4, enlarged cross-sectional views of the thread portions 30, 32 can be seen. In a preferred embodiment, the thread angle a of the thread portions 30, 32 is preferably 25°, but can be between 20°-30°. It has been determined that this range of thread angles is optimal for purchasing in the different regions of the bone tissue. The radius β of the thread portion 30, 32 is preferably 0.5 mm, but can be between 0.1 mm-1 mm. Again, it has been determined that this range for the radius is optimal for purchasing in the different regions of the bone tissue.

Turning back to FIGS. 1-4, the thread portions 30, 32 on the shaft 28 of the screw 10 is preferably a multi-start thread. More specifically, in a preferred embodiment, thread portions 30, 32 of the bone screw 10 is a two-start thread. Multi-start threads have the advantage of providing a thread on a screw shaft that has a smaller thread pitch (discussed below) than would be the case if the thread is a single-start thread. A smaller thread pitch can enhance the security of the fixation in bone tissue as well as increase the rate of installation of the screw in the bone tissue. In other preferred embodiments, a single-start thread portion as well as three or more start thread portion is also contemplated.

As mentioned above, the thread pitch is defined as the distance along the axis of the screw between adjacent thread peaks, shown in FIG. 2 as y. The thread lead is defined as the distance that is travelled along the axis of the screw in one complete 360° revolution of the screw, shown in FIG. 2 as x. In a preferred embodiment, the number of starts of the thread portions 30, 32 is equal to the ratio of the thread lead x to the thread pitch y. For example, for a two-start thread, the thread lead is preferably 5 mm and the thread pitch is preferably 2.5 mm. In a preferred embodiment, the bone screw 10 includes a two start thread. In another preferred embodiment, the thread pitch y is substantially constant over thread portions 30, 32.

Dimensions, in millimeters, of a preferred embodiment of the bone screw 10, which is suitable for use as a bone screw in a vertebral body, are as follows:

| L | L1 | L2 |
|---|---|---|
| 30.0 | 15.0 | 15.0 |
| 35.0 | 20.0 | 15.0 |
| 40.0 | 20.0 | 20.0 |
| 45.0 | 25.0 | 20.0 |
| 50.0 | 25.0 | 25.0 |
| 55.0 | 30.0 | 25.0 |
| 60.0 | 35.0 | 25.0 |
| 65.0 | 40.0 | 25.0 |
| 70.0 | 45.0 | 25.0 |
| 75.0 | 50.0 | 25.0 |
| 80.0 | 55.0 | 25.0 |
| 85.0 | 60.0 | 25.0 |
| 90.0 | 65.0 | 25.0 |
| 95.0 | 70.0 | 25.0 |
| 100.0 | 75.0 | 25.0 |

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A bone screw for surgical implantation into bone tissue, comprising:
a head portion, the head portion having a generally spherical shape and a recess for receiving an end of a driving instrument;
a shank portion, the shank portion comprising a shaft having a proximal end and a distal end; and
a neck portion, the neck portion having a proximal end and a distal end, the proximal end of the neck portion connects to the head portion and the distal end of the neck portion connects to the shank portion;
wherein at least a portion of the proximal end of the shaft is surrounded by a first thread portion and at least a portion of the distal end of the shaft is surrounded by a second thread portion,
wherein the first thread portion has a substantially constant diameter and the second thread portion has a substantially constant diameter, and
wherein the diameter of the first thread portion is greater than the diameter of the second thread portion.

2. The bone screw of claim 1, wherein the first thread portion has a first pitch and the second thread portion has a second pitch and wherein the first pitch of the first thread portion is substantially equal to the second pitch of the second thread portion.

3. The bone screw of claim 1, wherein at least a portion of the head portion has texturing.

4. The bone screw of claim 1, wherein the neck portion comprises a generally cylindrical portion coupled to a frustoconical portion, the frustoconical portion having a proximal end and a distal end,
wherein the diameter of the generally cylindrical portion is substantially equal to a minor diameter of the screw, and
wherein the diameter of the proximal end of the frustoconical portion is substantially equal to the diameter of the generally cylindrical portion and the diameter of the distal end of the frustoconical portion is substantially equal to a major diameter of the screw.

5. The bone screw of claim 1, wherein the first and second thread portions are a multi-start thread.

6. The bone screw of claim 1, wherein the first and second thread portions are a single-start thread.

7. The bone screw of claim 1, wherein the diameter of the shaft is substantially constant from the proximal end to the distal end.

8. The bone screw of claim 1, wherein the first and second thread portions are any one of the following threads types: Modified Buttress, Buttress, Acme, Unified, Whitworth and B&S Worm.

9. The bone screw of claim 1, wherein the screw is made from stainless steel alloys, titanium, titanium based alloys, or polymeric materials.

10. The bone screw of claim 1, wherein the ratio of the diameter of the first thread portion to the ratio of the second thread portion is approximately 1.0 to 1.5.

11. The bone screw of claim 1, wherein the first and second thread portions have a thread angle that is between approximately 20° to 30°.

12. The bone screw of claim 1, wherein the first and second thread portions have a radius that is between approximately 0.1 mm to 1.0 mm.

13. A bone screw for surgical implantation into a vertebral body having a cancellous region and a cortical region, comprising:
a head portion, the head portion having a generally spherical shape and a recess for receiving an end of a driving instrument;
a shank portion, the shank portion comprising a shaft having a proximal end and a distal end; and
a neck portion, the neck portion having a proximal end and a distal end, the proximal end of the neck portion connects to the head portion and the distal end of the neck portion connects to the shank portion;
wherein at least a portion of the proximal end of the shaft is surrounded by a first thread portion and at least a portion of the distal end of the shaft is surrounded by a second thread portion,
wherein the diameter of the first thread portion is greater than the diameter of the second thread portion, and
wherein the larger diameter of the first thread portion includes a larger purchasing surface area to grip the bony tissue in the cancellous region of the vertebral body and the smaller diameter of the second thread portion includes a smaller purchasing surface area to grip the bony tissue in the cortical region of the vertebral body.

14. A bone screw for surgical implantation into bone tissue, comprising:
a head portion, the head portion having a generally spherical shape with an outer surface, a recess for receiving an end of a driving instrument, and texturing on at least a portion of the outer surface;
a shank portion, the shank portion comprising a shaft having a proximal end, a distal end, and a distal tip, the diameter of the shaft is substantially constant from the proximal end to the distal end and decreases from the distal end to the distal tip; and
a neck portion, the neck portion having a proximal end and a distal end, the proximal end of the neck portion connects to the head portion and the distal end of the neck portion connects to the shank portion;
wherein at least a portion of the proximal end of the shaft is surrounded by a first thread portion and at least a portion of the distal end of the shaft is surrounded by a second thread portion,
wherein the first thread portion has a substantially constant diameter and the second thread portion has a substantially constant diameter,
wherein the diameter of the first thread portion is greater than the diameter of the second thread portion,
wherein between the first thread portion and the second thread portion there is a transition region where is diameter of the thread decreases gradually from the first thread portion diameter to the second thread portion, and
wherein the first thread portion has a first pitch and the second thread portion has a second pitch and wherein the first pitch of the first thread portion is substantially equal to the second pitch of the second thread portion.

15. The bone screw of claim 14, wherein the neck portion comprises a generally cylindrical portion coupled to a frustoconical portion, the frustoconical portion having a proximal end and a distal end,
wherein the diameter of the generally cylindrical portion is substantially equal to a minor diameter of the screw, and
wherein the diameter of the proximal end of the frustoconical portion is substantially equal to the diameter of the generally cylindrical portion and the diameter of the distal end of the frustoconical portion is substantially equal to a major diameter of the screw.

16. The bone screw of claim 14, wherein the bone screw is a self starting screw.

17. The bone screw of claim 14, wherein the screw is made from stainless steel alloys, titanium, titanium based alloys, or polymeric materials.

18. The bone screw of claim 14, wherein the ratio of the diameter of the first thread portion to the ratio of the second thread portion is approximately 1.0 to 1.5.

19. The bone screw of claim 14, wherein the first and second thread portions have a thread angle that is between approximately 20° to 30°.

20. The bone screw of claim 14, wherein the first and second thread portions have a radius that is between approximately 0.1 mm to 1.0 mm.
